(19) United States
(12) Patent Application Publication
(10) Pub. No.: US 2020/0177974 A1
(43) Pub. Date: Jun. 4, 2020

(54) ENVIRONMENT MONITORING DEVICE

(71) Applicants: HONG FU JIN PRECISION INDUSTRY (WuHan) CO., LTD., Wuhan (CN); HON HAI PRECISION INDUSTRY CO., LTD., New Taipei (TW)

(72) Inventors: CHUN-HSIEN CHO, New Taipei (TW); LI-TING CAI, Houston, TX (US)

(21) Appl. No.: 16/395,524

(22) Filed: Apr. 26, 2019

(30) Foreign Application Priority Data
Dec. 3, 2018 (CN) 201811467632.3

(51) Int. Cl. H04Q 9/00; G01L 19/00; G01N 33/00; G01J 5/02; G01J 1/42; H04L 29/08

(52) U.S. Cl. CPC H04Q 9/00; G01L 19/0092; G01N 33/004; H04L 67/12; G01J 5/02; G01J 1/42; G01N 33/0047

(57) ABSTRACT

An environment monitoring device includes a sensing unit, a communication unit, a processor, and a storage unit. The sensing unit includes a number of sensors for sensing environmental parameters in real time. The storage unit stores preset standard values of the environmental parameters. The processor is configured to acquire the environmental parameter data sensed by the sensors and transmit the environmental parameter data to the storage unit or transmit these data to terminal device by means of the communication unit, fit a trend line on a graph according to the acquired environmental parameter data, and determine whether the acquired environmental parameter data exceeds a preset standard value.

### FIELD

The subject matter herein generally relates to environment monitoring devices, and more particularly to an environment monitoring device for monitoring a plurality of environmental parameters.

### BACKGROUND

Currently, in order to monitor environmental parameters, multiple types of sensors need to be set up to detect different environmental parameters. Current environmental monitoring methods may not be efficient and require lots of equipment.

### BRIEF DESCRIPTION OF THE DRAWINGS

Implementations of the present disclosure will now be described, by way of embodiments, with reference to the attached figures.

FIG. 1 is a block diagram of a first embodiment of an environment monitoring device.

FIG. 2 is a block diagram of a monitoring and integration system implemented in the environment monitoring device.

FIG. 3 is a block diagram of a second embodiment of the environmental monitoring device.

### DETAILED DESCRIPTION

It will be appreciated that for simplicity and clarity of illustration, where appropriate, reference numerals have been repeated among the different figures to indicate corresponding or analogous elements. Additionally, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “coupled” is defined as connected, whether directly or indirectly through intervening components, and is not necessarily limited to physical connections. The connection can be such that the objects are permanently connected or releasably connected. The term “comprising” means “including, but not necessarily limited to”; it specifically indicates open-ended inclusion or membership in a so-described combination, group, series and the like.

In general, the word “module” as used hereinafter refers to logic embodied in hardware or firmware, or to a collection of software instructions, written in a programming language such as, for example, Java, C, or assembly. One or more software instructions in the modules may be embedded in firmware such as in an erasable-programmable read-only memory (EPROM). It will be appreciated that the modules may comprise connected logic units, such as gates and flip-flops, and may comprise programmable units, such as programmable gate arrays or processors. The modules described herein may be implemented as either software and/or hardware modules and may be stored in any type of computer-readable medium or other computer storage device.

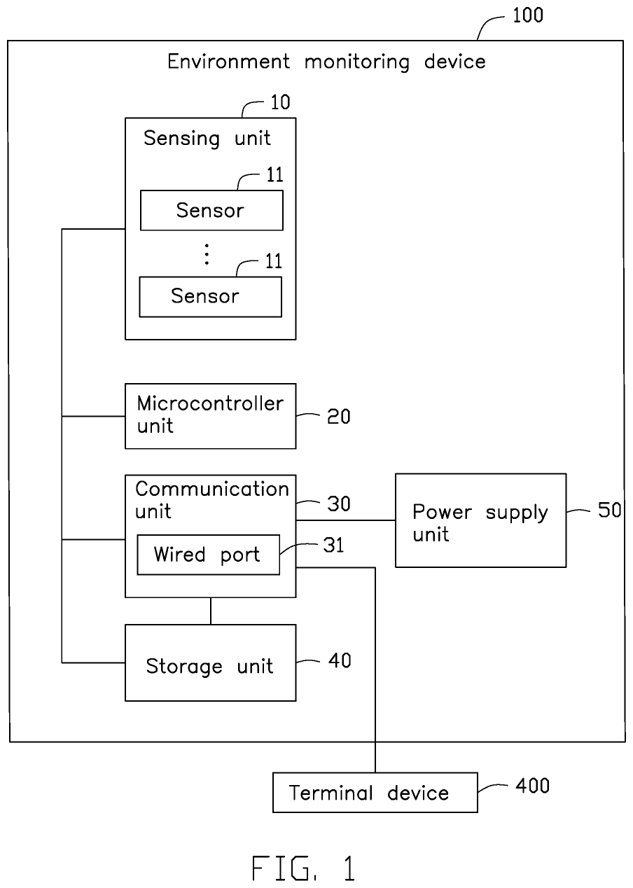

FIG. 1 shows a first embodiment of an environment monitoring device 100, which includes a sensing unit 10, a processor 20, a communication unit 30, and a storage unit 40. The sensing unit 10, the processor 20, the communication unit 30, and the storage unit 40 are electrically coupled together. It should be understood that FIG. 1 does not illustrate every component of the environment monitoring device 100, and the environment monitoring device 100 may include other components not described herein.

The sensing unit 10 includes a plurality of sensors 11 for sensing a plurality of environmental parameters in real time. The plurality of sensors 11 include a temperature sensor for sensing temperature, a humidity sensor for sensing humidity, a carbon monoxide sensor for sensing a concentration of carbon monoxide, a carbon dioxide sensor for sensing a concentration of carbon dioxide, a volatile organic substance sensor for sensing a concentration of volatile organic substances, a light intensity sensor for sensing an intensity of light, an infrared sensor for sensing infrared light, and a dynamic sensor for sensing a presence, distance, and speed of objects. It can be understood that the sensor 11 is not limited to the above-mentioned sensors, and other types of sensors, such as a barometric sensor, a smoke sensor, an image sensor, or the like may be provided according to actual needs.

The processor 20 is configured to process environmental parameter data and execute a plurality of instructions. The processor 20 can be a central processing unit, a digital signal processor, a processor on a chip, or the like.

The communication unit 30 includes a wired port 31. The wired port 31 can be a wired communication port such as a USB, a Serial Peripheral Interface (SPI), a Universal Asynchronous Receiver/Transmitter (UART), or a Local Area Network (LAN).

The communication unit 30 can be coupled to a terminal device 400 to upload the environmental parameter data sensed by the sensing unit 10 and processed by the processor 20 to the terminal device 400.

The storage unit 40 is configured to store the environmental parameter data and a plurality of instructions, which are executed by the processor 20 to perform corresponding functions. The storage unit 40 is further configured to store preset standard values of environmental parameters.

The storage unit 40 can be a hard disk, a universal serial bus (USB) drive, a random access memory, or the like.

In at least one embodiment, the storage unit 40 can be an internal memory system, such as a flash memory, a random access memory (RAM), a read-only memory (ROM), or the like.

The environment monitoring device 100 further includes a power supply unit 50. The power supply unit 50 is coupled to the communication unit 30 for supplying power to the sensing unit 10, the processor 20, the communication unit 30, and the storage unit 40.

In at least one embodiment, the power supply unit 50 is a direct current (DC) power storage device, such as a battery.

In at least one embodiment, the power supply unit 50 is a DC/DC or alternating current (AC)/DC power supply.

When the communication unit 30 is a USB drive, the communication unit 30 can be coupled to the terminal device 400 to supply power to the sensing unit 10, the processor 20, and the storage unit 40.

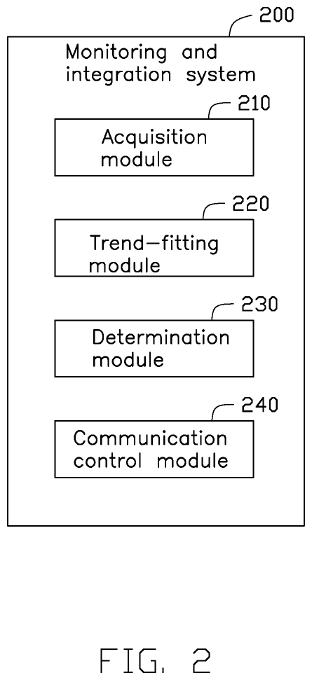

Referring to FIG. 2, a monitoring and integration system 200 applied in the environment monitoring device 100 includes an acquisition module 210, a trend-fitting module 220, a determination module 230, and a communication control module 240. In one embodiment, the acquisition module 210, the trend-fitting module, the determination module 230, and the communication control module 240 are a plurality of instructions stored in the storage unit 40 and executed by the processor 20 to perform corresponding functions. In other embodiments, the acquisition module 210, the trend-fitting module, the determination module 230, and the communication control module 240 may be firmware embedded in the processor 20.

The acquisition module 210 is configured to acquire the environmental parameter data sensed by the plurality of sensors 11 and transmit the environmental parameter data to the processor 20 and the storage unit 40. The trend-fitting module 220 is configured to fit a trend line on a graph according to the acquired environmental parameter data. The determination module 230 is configured to determine whether the acquired environmental parameter data exceeds a preset standard value. The communication control module 240 is configured to control the communication unit 30 to transmit the environmental parameter data to the terminal device 400.

In one embodiment, if any of the acquired environmental parameter data exceeds the preset standard value, the communication control module 240 sends a prompt to the terminal device 400 to alert that the acquired environmental parameter data exceeds the preset standard value.

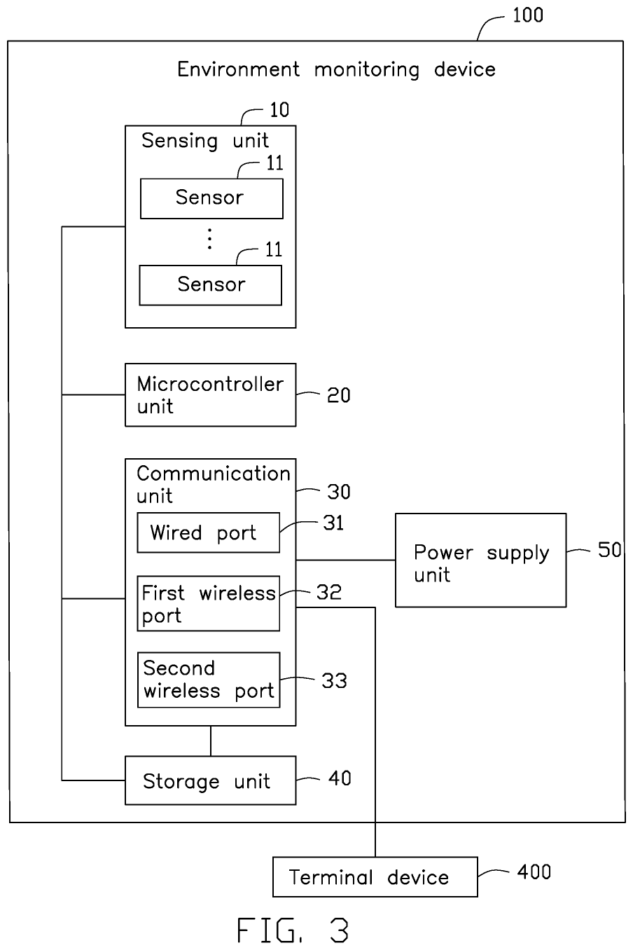

FIG. 3 shows an environment monitoring device 100 according to a second embodiment. The second embodiment is different from the first embodiment in that the communication unit 30 further includes a first wireless port 32 and a second wireless port. 33. The first wireless port 32 is configured to wirelessly couple the communication unit 30 to the terminal device 400. The second wireless port 33 is configured to wirelessly coupled the communication unit 30 to an external device, such as an alarm device, a purification device, a display, or the like. The first wireless port 32 and the second wireless port 33 may be at least one of a WiFi, BLUETOOTH, ZigBee, Z-wave Alliance, or other suitable wireless communication port.

The environmental monitoring device 100 of both embodiments can integrate the environmental parameter data of the plurality of environmental parameters. Thus, it is convenient to monitor various environmental parameters in real time, thereby saving monitoring costs.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms used in the claims.

What is claimed is:

1. An environment monitoring device for monitoring environmental parameters, the environment monitoring device comprising:
a sensing unit comprising a plurality of sensors for sensing a plurality of environmental parameters in real time;
a communication unit configured to be coupled to a terminal device;
a processor; and
a storage unit configured to store preset standard values of environmental parameters and a plurality of instructions, which when executed by the processor, causes the processor to:
acquire the environmental parameter data sensed by the plurality of sensors and transmit the environmental parameter data to the storage unit or transmit these data to terminal device by means of the communication unit;
fit a trend line on a graph according to the acquired environmental parameter data; and
determine whether the acquired environmental parameter data exceeds a preset standard value.

2. The environment monitoring device of claim 1, wherein:
the plurality of sensors comprise a temperature sensor for sensing temperature, a humidity sensor for sensing humidity, and a light intensity sensor for sensing an intensity of light.

3. The environment monitoring device of claim 2, wherein:
the plurality of sensors further comprise a carbon monoxide sensor for sensing a concentration of carbon monoxide, a carbon dioxide sensor for sensing a concentration of carbon dioxide, and a volatile organic substance sensor for sensing a concentration of volatile organic substances.

4. The environment monitoring device of claim 3, wherein:
the plurality of sensors further comprise an infrared sensor for sensing infrared light.

5. The environment monitoring device of claim 4, wherein:
the plurality of sensors further comprise a dynamic sensor for sensing a presence, distance, and speed of objects.

6. The environment monitoring device of claim 5, wherein:
the plurality of sensors further comprise a barometric sensor, a smoke sensor, and an image sensor;
the image sensor is configured to detect environmental images.

7. The environment monitoring device of claim 1, wherein:
the communication unit comprises a wired port;
the wired port is a universal serial bus (USB) port, a serial peripheral interface port, a universal asynchronous transceiver port, or a local area network port.

8. The environment monitoring device of claim 7, wherein:
the communication unit further comprises a first wireless port;
the first wireless port is a WiFi port, a BLUETOOTH port, a ZigBee port, or a Z-wave Alliance port.

9. The environment monitoring device of claim 8, wherein:
the communication unit further comprises a second wireless port using for communication with other wireless device;
the second wireless port is a WiFi port, a BLUETOOTH port, a ZigBee port, or a Z-wave Alliance port.

10. The environment monitoring device of claim 1 further comprising a power supply unit configured to provide power to the sensing unit, the microcontroller unit, the communication unit, and the storage unit.

11. The environment monitoring device of claim 10, wherein:
the power supply unit is a direct current power storage device.

12. An environment monitoring method for monitoring environmental parameters, the environment monitoring method comprising:
acquiring, by a plurality of sensors, environmental parameter data and transmitting the environmental parameter data to the microcontroller unit and the storage unit;
fitting a trend line on a graph according to the acquired environmental parameter data; and
determining whether the acquired environmental parameter data exceeds a preset standard value.

13. The environment monitoring method of claim 12 further comprising:
transmitting the environmental parameter data to a terminal device.

14. The environment monitoring method of claim 13 further comprising:
sending a prompt to the terminal device if the acquired environmental parameter data exceeds the preset standard value.
